(19) United States
(12) Patent Application Publication
(10) Pub. No.: US 2006/0020272 A1
(43) Pub. Date: Jan. 26, 2006

(54) Semi-robotic suturing device

(76) Inventor: Philip L. Gildenberg, Houston, TX (US)

Correspondence Address: VINSON & ELKINS L.L.P., 1001 FANNIN STREET, 2300 FIRST CITY TOWER, HOUSTON, TX, 77002-6760, US

(21) Appl. No.: 11/166,552

(22) Filed: Jun. 24, 2005

Related U.S. Application Data
(60) Provisional application No. 60/582,757, filed on Jun. 24, 2004.

(51) Int. Cl. A61B 17/04

(52) U.S. Cl. 606/144

(57) ABSTRACT

A semi-robotic apparatus and methods of use thereof for suturing body tissue, wherein the apparatus includes a housing; at least two distal arms connected to and extending distally from the housing, wherein the at least two distal arms are independently both extendable and retractable; a suture needle clasp connected to a distal end of each of the at least two distal arms, wherein the suture needle clasp is radially rotateable orthogonal to the longitudinal axis of the distal arm to which it is connected; and at least one controller operable for controlling at least a portion of the extension or retraction of the at least two distal arms, the rotation of the suture clasps and the opening and closing of the suture needle clasps.

### REFERENCE TO RELATED APPLICATION

This application claims priority to U.S. Provisional application No. 60/582,757, filed Jun. 24, 2004.

### BACKGROUND OF THE INVENTION

1. Field of the Invention

This invention relates to a surgical apparatus for suturing tissue, and more particularly to a semi-robotic suturing device that is useful in the suturing of tissue. The invention of the present disclosure is particularly helpful for the suturing of tissue within a confined space or with small suture needles. The invention disclosed also provides a mechanism for optimizing the trajectory of a suture needle as it pierces and passes through the tissue to be sutured in order to minimize trauma to the tissue.

2. Description of Related Art

During many medical procedures, the suturing of tissue can be one of the most time consuming and tedious elements. Suturing ordinarily involves the physician holding an instrument in each hand. The tissue forceps alternately grasps the tissue and the needle, leaving no instrument free to hold the tissue together throughout the suturing process. For example, suturing of tissue by a right handed surgeon typically involves a needle holder being held in the right hand of a physician and a pair of forceps in the left. The suture needle is grasped in a needle holder with the right hand, while the tissue is initially grasped by forceps in the left hand. The needle is then used to pierce the tissue and pushed through the tissue until the needle holder is adjacent to the tissue. The tissue is then released from the forceps in the left hand and the distal end of the needle is grasped by the forceps. The needle is then released from the needle holder in the right hand and pulled through the tissue with the forceps. The base of the needle is then grasped again by the needle holder in the physician's right hand and the needle is released from the forceps in the left hand. The suture is then pulled the rest of the way through the tissue until the proper tension holds the tissue together. The forceps are then used to grasp the tissue again in preparation for the next insertion of the suture needle.

Often, the suturing of tissue must be performed in a limited or confined space, such as within a body cavity, through a surgical opening in the body wall, or through an endoscope or endoscopic working channel. In these instances, the suturing procedure is made even more difficult because of limited mobility and a potentially limited field of view. Furthermore, the restriction of mobility and view increases the possibility of dropping or improperly placing the suture needle during those portions of the suturing procedure in which the needle is transferred from needle holder to forceps and back again. In order to alleviate or reduce some of these difficulties, suturing aids such as the one described in U.S. Pat. No. 5,938,668 have been developed. The instrument disclosed therein provides the physician with increased certainty with regard to the positioning, release, and recapturing of the suturing needle by providing jaws on the distal ends of two elongated tubular members. These jaws are controllable in such a fashion as to allow one set of jaws to grasp the suture needle, while the other set is retracted toward a handle (housing). The tissue to be sutured is then pierced and the suture needle passed though the tissue until its distal end is clear of the tissue. The retracted member is then extended and the jaws at its distal end engage the suture needle. The jaws of the other member then release the suture needle and retract proximally toward the handle. Therefore, this mechanism allows for the passing of the suture needle between two sets of jaws within a restricted area, while providing the security of always having physical control of the needle itself, as well as the tissue.

The advantages provided by such devices, however, are not limited to suturing in a confined space. Many types of surgical procedures, such as microvascular anastemosis require the use of extremely small suturing needles. The automatic transfer of a small suture needle from one jaw to another decreases the possibility of the needle being dropped or misgrasped due to is small size. Furthermore, this automatic transfer will allow the physician to maintain his or her viewing focal point on the tissue being ligated instead of having to switch such focal point back and forth between an instrument in either hand and the tissue itself. Finally, such devices allow the physician to essentially suture with one hand, thereby, enabling the physician to use the other hand to continually stabilize the tissue thus allowing for a more precise suture placement. The possibility of increased stabilization of the tissue being sutured and more precise suture placement is advantageous for suturing tissues such as suturing multiple layers of tissue, suturing thin-walled blood vessels, or suturing tissues that are under traction or tension that are susceptible to damage from distortion introduced through the movement of the suture needle.

As discussed above, in a typical suturing procedure, the tissue is pierced by the suturing needle followed by the needle being passed through the tissue and grasped from the other side where it is pulled the rest of the way through and out of the tissue. The passing of the suturing needle through the tissue is controlled by the force exerted on the needle through the needle holder or through rotation of the suturing device. However, because every suturing needle, by its physical nature, has a given length and arc, the physician must attempt to mimic that arc as the needle passes through the tissue for the length of the needle in order to minimize distortion of the tissue while placing the suture. Adding to this complexity is the fact the suturing needles come in a wide variety of lengths and arcs.

A further mechanical disadvantage occurs because the needle holders commonly used do not hold the needle at the center of rotation of the normal wrist, but sweep the needle through an arc displaced several centimeters from the center of rotation of the surgeon's wrist, so that the surgeon must artificially provide compensatory movement to move the needle smoothly through its arc, which is a function of the needle size and curvature. Furthermore, even suturing aids such as the device described above do not utilize jaws or suture clasps that adjust to the angle/arc of the suture needle. This lack of adjustment increases the difficulty of maintaining the proper arc of needle passage by increasing the deviation between the center of rotation for the suture needle and the center of rotation for the device.

It would, therefore, be advantageous to have a suturing device that was capable of continually maintaining physical control of a suturing needle while simultaneously providing a mechanism for driving the suturing needle through the tissue along the arc defined by the needle itself. In addition, such a device would be particularly useful if it could be utilized with any number of the wide variety of suturing needles available. Alternatively, it may be advantageous to have several sizes of the semi-robotic/robotic suturing device to accommodate all sizes of suturing needles from those used in microvascular or endoscopic procedures to those used to suture large vessels or heart valves.

### SUMMARY OF THE INVENTION

A semi-robotic apparatus for suturing body tissue including: a housing; at least two distal arms connected to and extending distally from the housing, wherein the at least two distal arms are independently both extendable and retractable; a suture needle clasp connected to a distal end of each of the at least two distal arms, wherein the suture needle clasp is radially rotateable orthogonal to the longitudinal axis of the distal arm to which it is connected; and at least one controller operable for controlling at least a portion of the extension or retraction of the at least two distal arms, the rotation of the suture clasps and the opening and closing of the suture needle clasps.

In certain embodiments, the semi-robotic apparatus, further includes a radial drive which rotates the at least two distal arms radially around the longitudinal axis of the housing which may be activated and deactivated by the at least one controller. In some of these embodiments, the rotation of the at least two distal arms radially around the longitudinal axis of the housing by the radial drive is at a predetermined continuous rate, where as in others, it is at a variable rate.

In certain other embodiments, the semi-robotic apparatus also includes a lateral drive which extends and retracts the at least two distal arms proximally and distally from the housing and a longitudinal drive which moves the at least two distal arms proximally and distally from the longitudinal center of the housing and rotates the at least two distal arms with respect to their longitudinal center. While in still other embodiments, the apparatus further includes a program interface, wherein the program interface can be used to store settings in the semi-robotic apparatus that direct the lateral positioning of the at least two distal arms by the lateral drive and the radial angle of the suture needle clasps by the longitudinal drive to match the arc of a predetermined suture needle.

In other embodiments, the semi-robotic apparatus also includes: a lateral drive which extends and retracts the at least two distal arms proximally and distally from the housing; a longitudinal drive which moves the at least two distal arms proximally and distally from the longitudinal center of the housing and rotates the at least two distal arms with respect to their longitudinal center; and a radial drive which rotates the at least two distal arms radially around the longitudinal axis of the housing. In some of these embodiments, the apparatus further includes a program interface, wherein the program interface can be used to store settings in the semi-robotic apparatus that direct the lateral positioning of the at least two distal arms by the lateral drive and the radial angle of the suture needle clasps by the longitudinal drive to match the arc of a predetermined suture needle. In still other of these embodiments, the rotation of the at least two distal arms radially around the longitudinal axis of the housing by the radial drive is at a predetermined continuous rate or at a variable rate.

Certain embodiments of the current invention are also functional with suture needles which have an arc that is not circular.

Certain other embodiments also include a gimble on which the at least two distal arms are mounted which allows the at least two distal arms to be offset at variable angles from the longitudinal axis of the housing.

Certain other embodiments of the semi-robotic apparatus also include an attachment for use by a robotic arm.

Still other embodiments of the present invention provide a semi-robotic suturing apparatus that includes: a housing; at least two suture clasping arms extending distally from the housing, wherein the at least two suture clasping arms comprise a suture clasping mechanism; a means for controlling the radial angle of the clasping mechanism with respect to the suture clasping arm; a means for controlling the independent extension distally from the handle or retraction proximally toward the handle of the retractable primary clasping arm or the retractable secondary clasping arm; and a means for independently controlling the clasping of a suture needle by the clasping mechanism of the retractable primary clasping arm or the clasping mechanism of the retractable secondary clasping arm.

The current invention also provides a method for suturing tissue with a semi-robotic suturing device which includes the steps of: providing a semi-robotic apparatus of the present invention, wherein a semi-robotic apparatus; using the at least one controller to direct: the clasping of a suture needle through the rotateable suture needle clasp connected to one of the distal arms; the retraction toward the housing of the other distal arms followed by its extension after the distal end of the suture needle has passed through the tissue to be sutured; the clasping of a suture needle through the rotateable suture needle clasp connected to the now extended other distal arm; the release of the suture needle from rotateable suture needle clasp of the first distal arm to engage the needle followed by the retraction of this distal arm proximally toward the housing.

### BRIEF DESCRIPTION OF THE FIGURES

This invention may be best understood by reference to the following description taken in conjunction with the accompanying drawings, in which like reference identify like elements, and in which:

FIG. 1 depicts one embodiment of the semi-robotic suturing device;

FIG. 2 depicts a longitudinal schematic of the semi-robotic suturing device suturing tissue;

FIG. 3 depicts the relationship between the coordinate positioning of the distal arms and the length and arc of various suture needles;

FIG. 4 demonstrates the relationship between the angular positioning of the suture needle clasps and the arc of the suture needle being utilized;

FIG. 5 displays the ability of the semi-robotic suturing apparatus to accommodate suture needles of varying arc;

FIG. 6 depicts the radial position of the distal arms of the robotic suturing apparatus from the longitudinal viewpoint, wherein the distal needle is grasped a short distance proximal to the point;

FIG. 7 shows various embodiments of the suture grasping clasps located at the end of the distal arms.

### DETAILED DESCRIPTION OF THE INVENTION

The present invention provides for a semi-robotic suturing device useful in the suturing of any type of tissue. Certain embodiments of the device are especially useful in suturing tissue within a restricted field, such as during endoscopic procedures, or through a small surgical opening. The device is also particularly useful when suturing with smaller suture needles, for instance, for microvascular anastemosis, in which the needle arc may have a diameter of only 3-4 mm, although the speed and ease of use as well as the decreased trauma to tissue would provide an advantage even with larger needles.

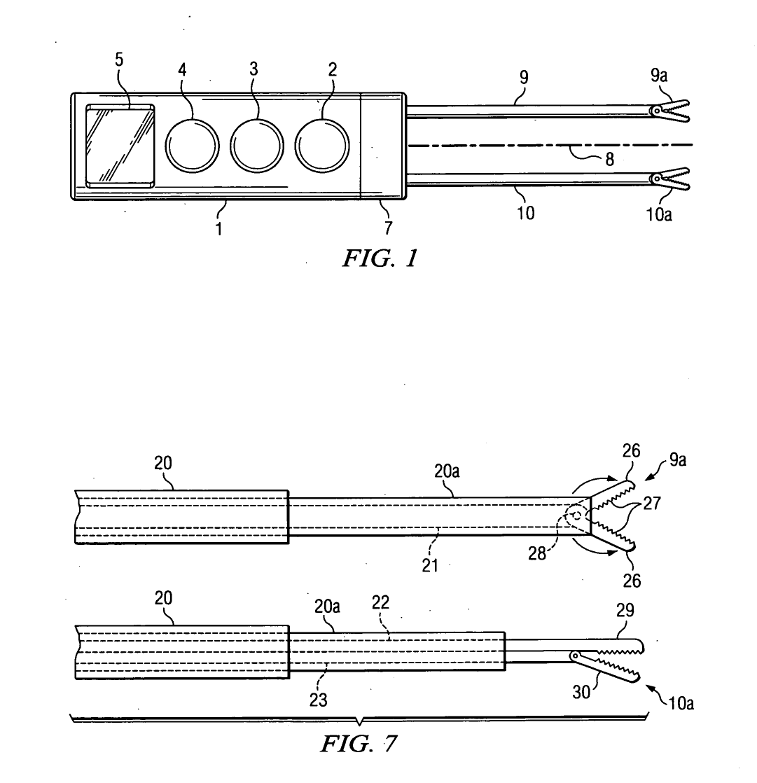

Referring to FIG. 1, a semi-robotic suturing device in accordance with one embodiment of the invention includes a housing 1 that may function as a handle for hand-held versions of the device or an attachment section for non-hand-held versions of the device, a set of at least one controllers 2-4, a program interface 5, and at least two distal arms 9,10 which are coupled either directly or indirectly to the housing 1. In certain embodiments, the distal arms may be adjusted to extend from the housing 1 at a defined angle and distance from the longitudinal center of the device 8. The distal arms 9,10 include suturing needle clasps 9a,10a at their distal most end. The controllers 2-4 located on the housing 1 of the robotic suture device may be actuated to cause the retraction or extension of a distal arm 9,10, the opening and closing of an individual suture needle clasp 9a or 10a, or the rotation of the distal arms 9,10 along a predefined arc (as discussed below).

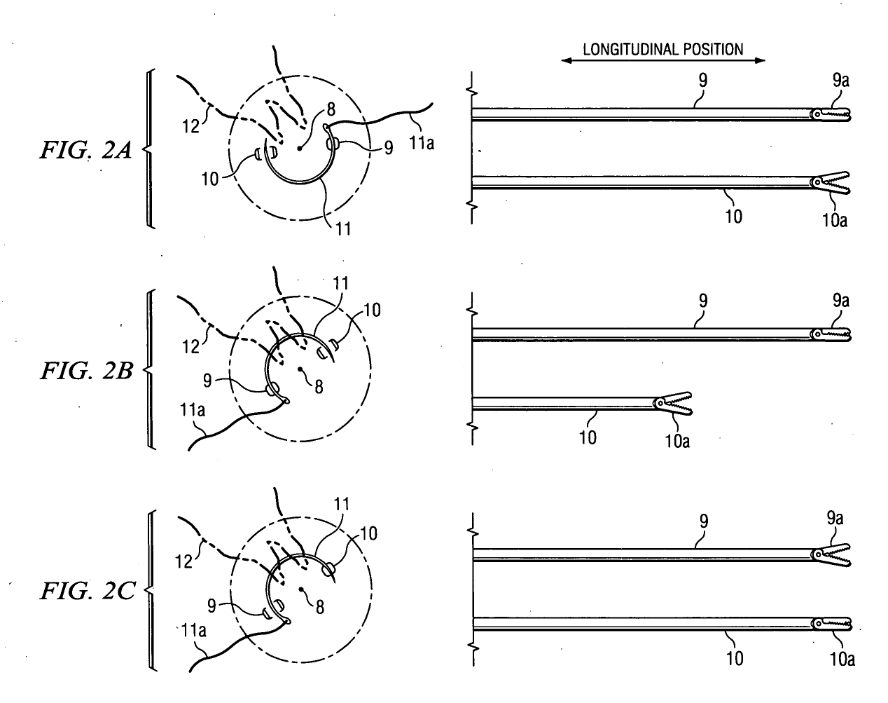

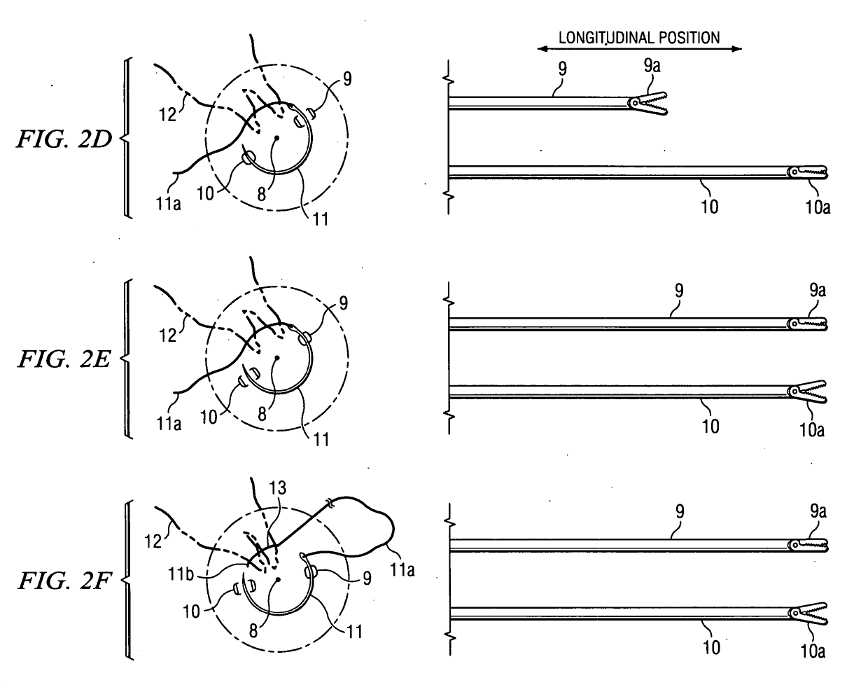

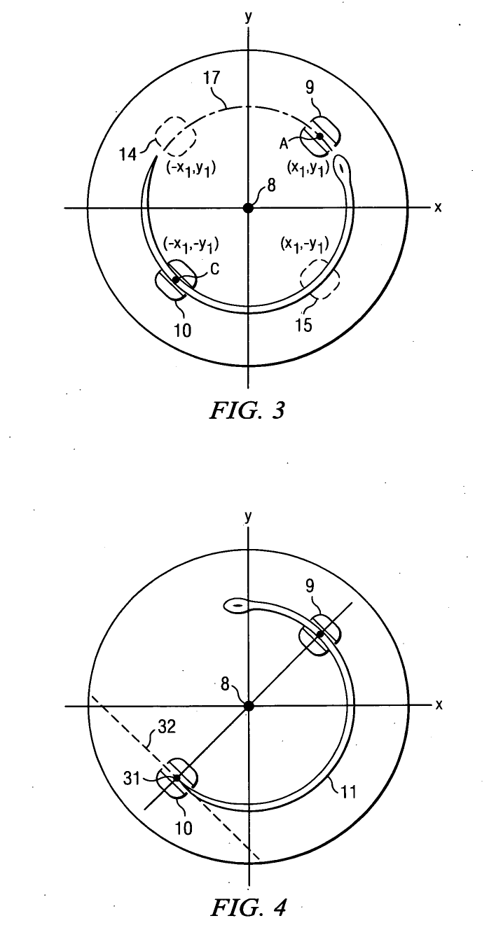

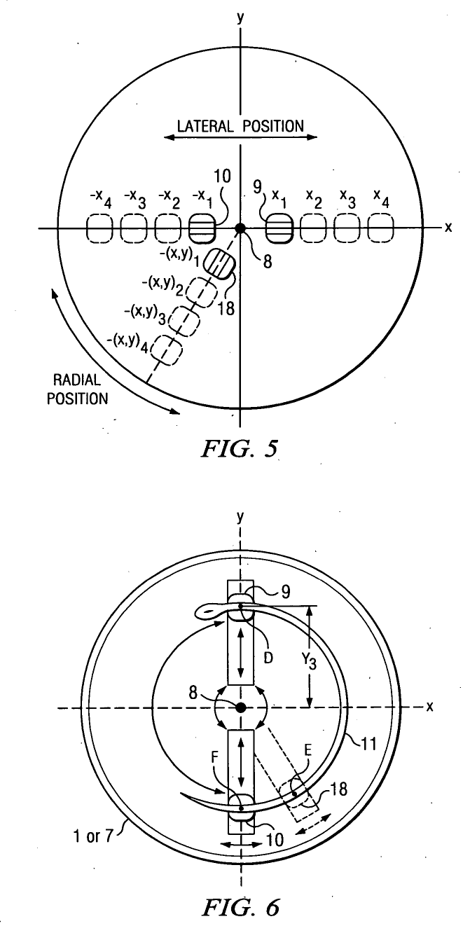

In certain embodiments, the housing 1 may enclose, wholly or partially, a lateral drive, a longitudinal drive and/or a radial drive. The lateral drive is capable of independently controlling the lateral position of each distal arm 9,10 with respect to the longitudinal center 8 of the device, as shown in FIG. 5. The longitudinal drive is capable of independently controlling the extension, distally away from the housing 1, or retraction, proximally toward the housing 1, of each distal arm 9,10, as shown in FIG. 2. The radial drive is capable of controlling the radial position of the distal arms 9,10 from one another (degrees separating the arms with the point of origin of the angle being the longitudinal center 8 of the semi-robotic suturing device or any other predetermined center of rotation, as shown in FIG. 5. The radial drive is also capable of rotating the distal arms 9,10 in a defined arc 17 around the longitudinal center 8 of the semi-robotic suturing device or any other predetermined center of rotation, as shown in FIG. 3. Alternate semi-robotic embodiments of the present invention may exclude the ability of the radial drive to rotate the distal arms 9,10 in order to move the suturing needle 11 through the desired arc 17 and rely on the physician to physically maneuver the device to do so.

TABLE 1
Individual steps for suturing correlated to FIG. 2 images.

| Steps | Distal Arm | | Suture Needle Clasp | | |
|---|---|---|---|---|---|
| | 9 | 10 | 9 | 10 | Activity |
| A | Extended | Extended/ | Closed | Open | Engage proximal end of needle |
| | | Retracted | | | |
| B | Extended | Retracted | Closed | Open | Push needle through tissue |
| B-C | Extended | Extended | Closed | Open | Position to engage needle |
| B-C | Extended | Extended | Closed | Closed | Both suture needle clasps engage needle |
| C | Extended | Extended | Open | Closed | Engage distal needle, release proximal needle |
| D | Retracted | Extended | Open | Closed | Suture needle clasp 9 set to clear tissue |
| D | Retracted | Extended | Open | Closed | Pull needle rest of way through tissue |
| D-E | Extended | Extended | Open | Closed | Position to engage needle |
| D-E | Extended | Extended | Closed | Closed | Both suture needle clasps engage needle |
| F | Extended | Extended | Closed | Open | Engage proximal needle, release distal needle |
| A | Extended | Retracted | Closed | Open | Suture needle clasp 10 set to clear tissue |

The present disclosure includes methods for using the semi-robotic suturing device. In one embodiment, the semi-robotic suturing device of the present invention can be manipulated through independent stages of the suturing cycle, as shown in FIG. 2. The needle may be loaded with both arms 9 and 10 extended, with both suture needle clasps at first open, then one suture needle clasps 10 disengages and its distal arm 10 is retracted—alternatively, the needle might be loaded with the device positioned as in FIG. 2B. One of skill in the art will readily recognize that the longitudinal position of the distal arms 9,10 (i.e., extended or retracted) is not critical for the loading of the needle and several possible positions would suffice for the initial loading of the suture needle. For example, a suturing cycle may be initiated with both distal arms being extended and a suture needle 11 loaded into the suture needle clasps 9a, 10a of the distal arms 9,10, termed the primary distal arm 9 (the other distal arm is termed the secondary distal arm 10) with the suture needle clasp 9a engaging/grasping the suture needle 11 near its proximal end, which is associated with the suture thread. The distal arms 9,10 are then inserted into the suturing field such that the distal tip of the suture needle 111 is adjacent to the tissue 12 to be sutured. In certain embodiments the semi-robotic suturing device can be positioned into the surgical cavity with both clamps of the suturing device engaged to protect the needle from contacting the tissue or being malaligned in the clamp by inadvertent contact with the tissue. The secondary distal arm 10 is then retracted as shown in FIG. 2B (although it could be retracted prior to loading the suture needle 11 or inserting the device into the suturing field) and the radial drive is activated to cause both distal arms 9,10 to rotate along an arc 17, which is defined by the length and shape of the suture needle 11 being used (as discussed below), causing the distal end of the suture needle 11 to pierce and move through the tissue 12. The radial drive may move the suture needle 11 to any position in which the distal end of the needle is clear of the tissue being sutured. As described above with respect to embodiments lacking the radial drive or in instances in which the radial drive is not activated, the physician may physically rotate the device in order to mimic the activity of the radial drive. The secondary distal arm 10 is then extended, as shown in FIG. 2C with the suture needle clasp 10a opened to engage the needle. The suture needle 11 is therefore engaged by both suture needle clasps 9a/10a with the pierced tissue between the clamps. The suture needle clasp 9a of the primary distal arm 9 is then opened to release the needle. The primary distal arm 9 is then retracted, as shown in FIG. 2D, and the radial drive is engaged to cause, or the physician causes, distal arms 9,10 to rotate again along an arc 17 which corresponds to the curvature of the suture needle 11, until the needle is free of the tissue. This rotation causes the proximal end of the needle to be pulled through the tissue being sutured bringing along with it the suture thread. The primary distal arm 9 is then extended longitudinally with the suture needle clasp 9a open, as shown in FIG. 2E, and the suture needle clasp 9a engages the needle at its proximal end. The suture needle clasp 10a of the secondary distal arm 10 then opens to disengage the needle and the device is pulled proximally away from the suturing field to obtain the proper tensions on the suture 11b. Alternatively, the tension maybe introduced immediately after the needle is pulled through the tissue and prior to it being transferred from the secondary suture needle clasp 10a to the primary suture needle clasp 9a, or the suture thread can be pulled through with a forceps or other instrument to secure proper tissue approximation and tension.

The device may be designed so the suture can be introduced by the surgeon's left hand or in the direction of a left-handed surgeon, in which case the roles of are 9 and 10 as described above would be reversed.

Because the tissue to be sutured is not always located tangentially to the direction in which the suturing device can be introduced into the incision, the distal end of the semi-robotic suturing device may be mounted on a hinge or gimbal so it may be angled by the surgeon to orient the suture tangential to the tissue through which the suture is to be thrust. Furthermore, in certain embodiments the radial drive maybe programmed to generate an enhanced initial thrust when causing the suture needle to pierce the tissue in order to increase the mechanical advantage of the needle over the tissue.

The use of the semi-robotic suturing device in such a procedure has several advantages over the typical suturing procedure. For instance, because the device enables the physician to complete the suturing process with one hand while a conventional set of forceps can be used by the other hand to stabilize the tissue being sutured the precision of the suture placement is increased and the distortion the tissue during the insertion of the suturing needle 11 is decreased. In addition, the semi-robotic suturing device never loses physical control over the suturing needle. In embodiments which include the radial drive, the device increases the precision of moving the suturing needle 11 through an arc that matches the arc 17 of the suturing needle thereby decreasing the distorting forces being imparted onto the tissue 12 by the force of the suture needle 11 being inserted and passed through. Furthermore, in embodiments which utilize the radial drive to move the suture needle 11, the rate of rotation may be variable. In other words, the device may be programmed through the program interface 5 to advance the suture needle 11 at a set constant speed or may be programmed to provide an increased initial thrust when piercing the tissue thereby increasing the suture needle's 11 ability to enter the tissue 12 while minimizing the tissue distortion created by its insertion. The distance the needle travels' through its arc can be accurately programmed to assure maximum travel of the needle through the tissue, while protecting the tissue against stress caused by pressure from the suture needle clasp 9a exerted by the suture needle clasp 9a advancing too far.

In certain embodiments of the present invention, the radial drive causes the distal arms 9,10 to travel along an arc 17 which is defined by the arc of the suture needle, as shown in FIG. 3. This arc may be centered around the longitudinal center of the device 8, while alternative embodiments of the present invention provide for the center of the arc 17 to be at a specified location other than the longitudinal center of the device. In other words, the center of the arc may be displaced from the center of the device. The center of the arc 17 and the size of the suture needle 11 will, however, still define or set the parameters for the radial path to be traveled by the distal arms 9,10.

The arc 17 to be traveled is defined by the curve of the suture needle 11 because every suture needle will have an optimal path or trajectory through the tissue being sutured that is directly related to the needle's arc or shape. FIG. 4 shows a diagram of the longitudinal view of the distal arm end of the device of the present invention. The trajectory of the suture needle 11 optimally will travel along an arc that is identical to the arc of the suture needle (at least for suture needles with an arc that represents a portion of a circle and the center of rotation within the arc of the needle defined by the length of the radius of that circle). If the suture needle 11 is moved along this arc 17, the area of intersection between the tissue and the needle should approximate the tangent point 31 between the arc 17 and a tangential vector that matches the inner surface of the suture needle clasps 9a, 110a, thereby decreasing or minimizing the amount of pulling/distorting introduced into the tissue by the suture needle as it pierces and passes through the tissue.

One of the significant differences between this device and the two-arm prior art is the configuration of the needle grasping part of the device. This device grasps across the curve of the needle, which holds it securely in its specific arc. The prior device grasped the needle from side to side, which would permit the needle to deviate from its arc with the slightest tissue pressure. Even if the needle is driven precisely along its arc, the tissue resistance would tend to cause it to move in relation to the jaws of the needle holder, which would cause it to advance through a path other than the arc of the needle, which would be far more likely with the prior device (only one of the advantages of this device over prior art).

Most suturing needles are defined by a curve that mirrors an arc of a circle, with the length commonly being ⅜ or ½ the circumference of that circle. Nevertheless, because suturing needles are available in a wide variety of shapes and sizes, the semi-robotic suturing device of the present invention is capable of being adjusted to configurations that will function with many different needles. The lateral and radial drives may be used to place the distal arms 9,10 at any necessary position within a Cartesian coordinate system, as shown in FIGS. 5 and 6. In other words, the lateral drive may be used to position the distal arms 9,10 at a predefined location along the arc which is determined by the suture needle to be used, while the radial drive can, likewise, be used to position the distal arms 9,10 at any point along that arc. For example, in FIG. 6, if the arc of the suturing needle 11 is circular and greater than 180 degrees, the distal arms 9,10 may be positioned at a location on the arc 180 degrees from each other and an equidistance from the center of rotation 8. Alternatively, if the suture needle 11 itself has an arc of less than 180 degrees, the radial drive may be used to position the distal arms 9,10 along the arc in a position less than 180 degrees apart to allow the distal arms 9,10 to interact with the needle. Alternatively, it may advantageous to use a suture needle of an arc slightly greater than 180 degrees, in which case the distal arms may be placed in positions along the arc greater than 180 degrees apart.

The semi-robotic suturing device of the present invention may also be used with suture needles having an elliptical or non-circular shaped arc as opposed to a circular one. In such cases, the distal arms 9,10 would be positioned by the radial and lateral drives along the elliptical arc defined by the suture needle 11. In such instances, the radial drive and lateral drive would work in concert to continually adjust the Cartesian coordinates of the two distal arms 9,10 during rotation such that their positions remain on the elliptical arc. Passing the suture needle 11 through the tissue 12 on an arc 17 that mimics the needle (circular or elliptical) is desirable because it will minimize any lateral or distal pulling and distortion of the tissue as it is being sutured.

In certain embodiments, the suture needle clasps will rotate to match the arc of the needle. In other words, when needles having greater or less than 180° of arc used, not only will the distal arms be moved to match the needles arc but the suture needle clasps will also rotate to match the needles arc, as shown in FIG. 6. For example, in certain embodiments of the present invention, the suture needle clasps 9a, 10a on the distal end of the distal arms 9,10 are radially positionable independent of the radial position of the arm, so that the x-y position of the arm, the length of the arm and the rotation of the arm may be adjusted independently. This feature allows the suture needle clasps 9a,10a to be placed in the optimal position for clasping the suture needle 11 regardless of the suture needle being used. FIG. 4 demonstrates that the bisecting vector of the suture needle clasp 9a,10a defined by the inner surface of each jaw 26 forms a line which is approximately tangential to the arc defined by the suture needle itself. In some embodiments, the tangent point 31 of contact between the tangential vector 32 and the arc defined by the suture needle 11 being used is in the center of the suture needle clasp 9a, 10a. The radial position of the suture needle clasp 9a,10a with respect to the distal arm 9,10 would therefore be such that each clasp is positioned in a manner that allows the tangential vector 32 defined by the inner surface of the clasp to intersect the arc defined by the suture needle at the tangent point 31. The positioning of the tangent point 31 in the center of the suture needle clasps 9a,10a increases the ability to maintain the proper positioning of the suture needle 11 when it is clasped through only one distal arm 9,10.

However, alternative embodiments of the present invention may allow for the tangent point 31 to be placed at a location within the suture needle clasp 9a,10a that is not in the center of the suture needle clasp 9a,10a. One of ordinary skill in the art would recognize that slight alterations in the positioning of the suture needle clasps 9a,10a (or the distal arms 9,10 for that matter) away from the described positions would still allow the device to function satisfactorily, especially in light of the fact that many tissues are elastic enough to accommodate the mis-positioning of the suture needle. In other words, slight to moderate deviations in the suture needle's 11 position or trajectory will not sufficiently impair the function or usefulness of the present invention and are therefore within the scope this disclosure.

Certain embodiments of the present invention provide for the semi-robotic suturing device to automatically adjust the positions of the distal arms 9,10 and the suture needle clasps 9a,10a, as well as the arc of rotation based on the particular suture needle to be used. The device may have multiple preprogrammed settings that correspond with various individual suture needles. For example, in certain embodiments the physician may simply enter a product number, or other unique identifier, for the suture needle to be used through the program interface 5 and the device will automatically assume the proper configuration, based on the stored information about the suture needle, allowing the device to advance the needle along the proper arc, piercing the tissue and passing throughout its length. Such programming may be contained within the device and have a means for entering the needle identifying data directly. Alternate embodiments provide for external programming of the device, such as linking the device to a computer, or other programming apparatus, through the program interface 5, thereby, allowing the desired configurations to be transmitted to the device. In the case of a suture needle with an elliptical arc, the program interface 5 may be used to input the course trajectory or set of coordinates as well as the suture needle clasp positions that are necessary to allow the device to move the suture needle along the prescribed arc.

The suture needle clasps 9a, 110a located on the distal end of the distal arms 9,10 may be of any design suitable for clasping a suture needle 11. One of ordinary skill in the art would understand that any number of mechanisms could be used to secure the suture needle. As such, the term suture needle clasp is meant to include all such mechanisms. For example, as shown in FIG. 7, the suture needle clasps 9a,10a may comprise a pair of jaws 26 similar to those found on a pair of forceps or ordinary needle holder. These jaws may be attached to a clasp control actuator 21 which is capable of being manipulated longitudinally with respect to a slideable portion 20a of a distal arm 9,10. The proximal movement of the clasp control actuator 21 with respect to the slideable portion 20a of a distal arm 9,10 may cause the hinge 28 connecting the two jaws 26 to be closed via mechanical force exerted on the exterior surface of the jaws by the interior surface of the slideable portion 20a of the distal arm 9,10 longitudinally along the length of the jaws 26. In certain embodiments, the device may contain a single hinge or a double action hinge mechanism for greater mechanical advantage, or other mechanism designed to assure firm grasp of the needle. In alternate embodiments the suture needle clasp, such as shown in FIG. 7, comprises a stationary jaw 29 connected to a clasp-control actuator 22 and a movable jaw 30 connected to a clasp-control actuator 23. This embodiment allows for the stationary jaw actuator 22 to remain in one position while the moveable jaw 30 having an angled portion may be moved distally away from the housing 1 of the device such that the angle captures the suture needle 11 by pinning it between the moveable jaw 30 and the stationary jaw 29. Furthermore, in some embodiments, the jaws may have a groove defining the position in which the needle is to be held in order to provide optimal orientation between the jaws and the needle. Such a groove may be shaped to correspond to the configuration of the cross-section of the part of the needle to be grasped, further insuring proper orientation of the needle.

Certain embodiments of the semi-robotic suturing device of the present invention further enable a physician to control each step of the suturing process. A set of controllers 2-4 (one or more controllers) located on the housing may be assigned a variety of related or independent functions. For example, in one embodiment a controller 2 may move the device forward through the suturing steps (wherein an individual step refers to any particular movement, such as a rotation of the distal arms 9,10, the extension or retraction of a distal arm 9,10, or the engaging or disengaging of a suture needle clasp 9a, 10a), while another controller 4 may move the device backward through the suturing steps and a third controller 3 might provide an emergency stop. In other embodiments two or more steps may be linked so as to occur sequentially upon activation of a single controller. For example, one input might cause the extension of a distal arm 9,10 followed by the engaging of its suture needle clasp 9a,10a. In alternate embodiments of the device may have a controller 2-4 which acts as an emergency release that can be toggled in either direction to release either one of the jaws selectively or can be depressed to release both simultaneously. Other embodiments of the device might provide a separate controller 2-4 for the extension and retraction of a given distal arm, the opening and closing of a particular suture needle clasp, and the forward and reverse rotation of the distal arms. While still other embodiments of the present invention may provide more or less controls than described above and one of skill in the art would readily recognize that multiple configurations for such controllers could adequately maneuver the device through the necessary steps of the suturing procedure.

The power source for the device may be either internal, contained within the device and battery operated or with a rechargeable power supply or may be external, connected to an external power source.

Finally, the semi-robotic suturing device of the present disclosure can be used manually by the physician holding it in his or her hand or the device can be mounted at the end of an automatically controlled long arm for endoscopic surgery (with the long arm being held by the physician) or robotically, with the position of the long arm controlled by the robot. If controlled robotically, the speed with which the needle is advanced may also be controlled by the robot to minimize tissue distortion.

What is claimed is:

1. A semi-robotic apparatus for suturing body tissue comprising:
a housing;
at least two distal arms connected to and extending distally from the housing, wherein the at least two distal arms are independently both extendable and retractable;
a suture needle clasp connected to a distal end of each of the at least two distal arms, wherein the suture needle clasp is radially rotateable orthogonal to the longitudinal axis of the distal arm to which it is connected; and
at least one controller operable for controlling at least a portion of the extension or retraction of the at least two distal arms, the rotation of the suture clasps and the opening and closing of the suture needle clasps.

2. The semi-robotic apparatus of claim 1, further comprising a radial drive which rotates the at least two distal arms radially around the longitudinal axis of the housing.

3. The semi-robotic apparatus of claim 2, wherein the radial drive can be activated and deactivated by the at least one controller.

4. The semi-robotic apparatus of claim 2, wherein the rotation of the at least two distal arms radially around the longitudinal axis of the housing by the radial drive is at a predetermined continuous rate.

5. The semi-robotic apparatus of claim 2, wherein the rotation of the at least two distal arms radially around the longitudinal axis of the housing by the radial drive is at a variable rate.

6. The semi-robotic apparatus of claim 1, further comprising:
a lateral drive which extends and retracts the at least two distal arms proximally and distally from the housing; and
a longitudinal drive which moves the at least two distal arms proximally and distally from the longitudinal center of the housing and rotates the at least two distal arms with respect to their longitudinal center.

7. The semi-robotic apparatus of claim 6, further comprising a program interface, wherein the program interface can be used to store settings in the semi-robotic apparatus that direct the lateral positioning of the at least two distal arms by the lateral drive and the radial angle of the suture needle clasps by the longitudinal drive to match the arc of a predetermined suture needle.

8. The semi-robotic apparatus of claim 1, further comprising:
a lateral drive which extends and retracts the at least two distal arms proximally and distally from the housing;
a longitudinal drive which moves the at least two distal arms proximally and distally from the longitudinal center of the housing and rotates the at least two distal arms with respect to their longitudinal center; and
a radial drive which rotates the at least two distal arms radially around the longitudinal axis of the housing.

9. The semi-robotic apparatus of claim 8, further comprising a program interface, wherein the program interface can be used to store settings in the semi-robotic apparatus that direct the lateral positioning of the at least two distal arms by the lateral drive and the radial angle of the suture needle clasps by the longitudinal drive to match the arc of a predetermined suture needle.

10. The semi-robotic apparatus of claim 9, wherein the suture needle arc is not circular.

11. The semi-robotic apparatus of claim 9, wherein the radial drive can be activated and deactivated by the at least one controller.

12. The semi-robotic apparatus of claim 9, wherein the rotation of the at least two distal arms radially around the longitudinal axis of the housing by the radial drive is at a predetermined continuous rate.

13. The semi-robotic apparatus of claim 9, wherein the rotation of the at least two distal arms radially around the longitudinal axis of the housing by the radial drive is at a variable rate.

14. The semi-robotic apparatus of claim 9, wherein the at least two distal arms are mounted on a gimble that allows the at least two distal arms to be offset at variable angles from the longitudinal axis of the housing.

15. The semi-robotic apparatus of claim 1, further comprising an attachment for use by a robotic arm.

16. A method for suturing tissue with a semi-robotic suturing device comprising:
providing a semi-robotic apparatus of claim 1, wherein the semi-robotic apparatus of claim 1 has two distal arms; and
using the at least one controller to direct:
the clasping of a suture needle through the rotateable suture needle clasp connected to one of the distal arms;
the retraction toward the housing of the other distal arms followed by its extension after the distal end of the suture needle has passed through the tissue to be sutured;
the clasping of a suture needle through the rotateable suture needle clasp connected to the now extended other distal arm;
the release of the suture needle from rotateable suture needle clasp of the first distal arm to engage the needle followed by the retraction of this distal arm proximally toward the housing.

17. The method of claim 16, wherein the semi-robotic apparatus of claim 1 further comprises:
a lateral drive which extends and retracts the at least two distal arms proximally and distally from the housing;
a longitudinal drive which moves the at least two distal arms proximally and distally from the longitudinal center of the housing and rotates the at least two distal arms with respect to their longitudinal center; and
a radial drive which rotates the at least two distal arms radially around the longitudinal axis of the housing.

18. The semi-robotic apparatus of claim 17, further comprising a program interface, wherein the program interface can be used to store settings in the semi-robotic apparatus that direct the lateral positioning of the at least two distal arms by the lateral drive and the radial angle of the suture needle clasps by the longitudinal drive to match the arc of a predetermined suture needle, or stored in a programming device.

19. The semi-robotic apparatus of claim 18, wherein the radial drive can be activated and deactivated by the at least one controller.

20. The semi-robotic apparatus of claim 19, wherein the rotation of the at least two distal arms radially around the longitudinal axis of the housing by the radial drive is at a predetermined continuous rate.

21. The semi-robotic apparatus of claim 19, wherein the rotation of the at least two distal arms radially around the longitudinal axis of the housing by the radial drive is at a variable rate.

22. A semi-robotic suturing apparatus comprising:
a housing;
at least two suture clasping arms extending distally from the housing, wherein the at least two suture clasping arms comprise a suture clasping mechanism;
a means for controlling the radial angle of the clasping mechanism with respect to the suture clasping arm;
a means for controlling the independent extension distally from the handle or retraction proximally toward the handle of the retractable primary clasping arm or the retractable secondary clasping arm;
a means for independently controlling the clasping of a suture needle by the clasping mechanism of the retractable primary clasping arm or the clasping mechanism of the retractable secondary clasping arm.
